(19) United States
(12) Patent Application Publication
(10) Pub. No.: US 2002/0124859 A1
(43) Pub. Date: Sep. 12, 2002

(54) Pigment composition

(75) Inventors: Krithivasan Bhagyalakshmi, Bangalore (IN); Indu Mani, Bangalore (IN); Hemant Sambhajiro Pawar, Ann Arbor, MI (US); Govindarajan Raman, Mumbai (IN)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/007,187

(22) Filed: Oct. 19, 2001

(30) Foreign Application Priority Data
Oct. 20, 2000 (IN) 940/MUM/2000
Jan. 22, 2001 (GB) 0101603.9

(51) Int. Cl. A61K 7/13

(52) U.S. Cl. 132/208

(57) ABSTRACT

Compositions for colouring hair and/or skin comprise:

(a) a cationic polymer;

(b) a natural phenol; and

(c) unripe tamarind extract.

### TECHNICAL FIELD

The present invention relates to compositions for changing the colour of keratinic fibers especially human hair. The composition can also be used to increase the colour of human skin. The composition is especially useful to obtain a range of desired colours on hair.

### BACKGROUND AND PRIOR ART

Melanins are naturally occurring pigments present in hair and skin and are synthesised in the melanocytes in presence of the enzyme tyrosinase. Two different melanins are found in human hair, the blonde, brown and black hair contain eumelanin while Pheomelanin is found in red hair. The enzyme tyrosinase catalyses the hydroxylation of tyrosine to 3,4-dihydroxyphenylalanine (DOPA) and its subsequent oxidation to dopachrome which further undergoes a series of complex reactions to form eumelanins and pheomelanins.

Cosmetic purposes has made the alteration of colouration of hair very popular. The discovery of synthetic hair dyes simplified the process of dyeing hair and gave much more reliable and versatile results in comparison the natural dyes. The preference to use natural materials is very high although this process has a lot of inconveniences and problems.

Aqueous hair dyeing compositions produced by reaction between DOPA and or a salt thereof with an inorganic oxidant such as soluble ammonium, sodium and potassium salts of ferricyanide and potassium permanganate in a buffered system with a pH range of 6 to 10, is reported (U.S. Pat. No. 5,279,618, Clairol Inc.).

U.S. Pat. No. 5,603,734 reports a method of permanently colouring hair by applying at least one catechol, a persulphate oxidising agent and a buffering system to provide a pH 7 to 11.

Use of quaternary amines along with compositions reported for colouring hair is known to improve the feel and combability of the hair after treatment (U.S. Pat. No. 4,402,700).

A novel natural hair colouring process described (Brown et.al., J of Soc. of Cos. Chemists, 48, 1997) relates to a process of converting DOPA on hair to 5,6-dihydroxyindole by careful selection of the oxidant. This indole is a precursor of black eumelanin and it is readily polymerised in situ to dye the hair black. They have also found that incorporation of sulfur-containing nucleophiles such as cysteine and other thiols into the above process produces a wide range of shades on hair.

The prior art relates to the use of inorganic oxidants for the production of melanin on hair and the synthetic hair colourants are toxic and cause allergic reactions in many people.

It has now been found that it is possible to colour hair using a natural plant extract in combination with a phenol and a metal ion. The use of the polymer along with the above mixture will help in improved deposition of the colour on the hair and also give other benefits like feel and combability etc. It is also possible to generate a wide range of shades by this process. The product will be safe as the composition is natural based. Since this product increases melanin synthesis, it could also be used on skin to increase skin colour and in tanning preparations.

Tamarindus is a monotypic genus of trees of the family Leguminosae represented by T.indica, indigenous to tropical Africa and South India. It is widely cultivated in the tropics and sub-tropics. The fruit of the tree is the most useful part which contains a sweetish, acidic pulp and hence used for souring of food products. It has now been found that a synergistic combination of the extract from the unripe fruits of this tree and a natural phenol can act as a hair dye.

### DESCRIPTION OF THE INVENTION

In one aspect, the present invention provides a composition for colouring hair and/or skin comprising:

(a) a cationic polymer;

(b) a natural phenol; and

(c) unripe tamarind extract.

According to one preferred aspect the present invention relates to a two component hair/skin colouring system comprising:

a. a cationic polymer 0.0005 to 0.05% by wt.

b. a natural phenol (weight range 50-500 mg/100 ml) and unripe tamarind extract (0.5-5.0 gm/100 ml) in a ratio 1:100 to 1:10 in a buffered system with a pH range of 4-10 containing 1-20 mg of a transition metal ion (Fe²⁺, Fe^{3+l , Cu}²⁺)

According to another aspect the present invention there is provided a method for dyeing hair comprising the steps of:

a) mixing a natural phenol, unripe tamarind extract and ferric chloride in a buffered system with a pH range of 4-10

b) applying an aqueous solution of the cationic polymer on to the hair

c) later applying the mixture described in (a)

d) allowing the said mixture to permanently colour the hair by remaining in contact with the hair for 8-16 hrs. Alternately step (d) can also be performed by intermittent applications of the mixture for 20 min time periods. Here at least 4-6 applications are recommended for best results.

The process of the invention may conveniently be practiced by providing pre-measured amounts of the reactants in separate containers packed in a kit form. It would be simple for the user to mix the components and subsequently spread the material on the hair to be dyed. This being a very simple operation can be carried out by anyone and does not require any professional expertise.

### DETAILED DESCRIPTION OF THE INVENTION

The hair/skin colouring composition of the present invention comprises an aqueous composition comprising a natural phenol and an extract from the unripe fruits of the plant Tamarindus indica. The dyeing of the hair is permanent and by the manipulation of the ingredients a range of colours can be achieved. By “permanent” one means that the colour is not removable by shampooing with standard surfactant based cosmetic shampoos.

Natural Phenol:

A wide variety of naturally occurring compounds having one or more phenolic hydroxyl groups, such as, for example, Catechol or DOPA species, can be utilized in the invention. It may include DOPA or its homologues, analogs or derivatives. It may be chosen from, for example, cysteinyl dopa, alpha alkyl dopa having 1-4, preferably 1 to 2 carbon atoms in the alkyl group, epinephrine, and dopa alkyl esters. Suitable soluble acid salts of dopa are selected from hydrochloride or sulphate salts. The suitable basic salts are the alkali metal salts or alkaline earth metal salts and sodium and potassium salts are preferred. Natural extracts of plants containing high levels (e.g., greater than 1% by weight, preferably greater than 10% by weight, more preferably greater than 50% by weight) of dopa and other catechols can also be used.

Buffering Agent:

The critical pH to be maintained is between 4 to 10 and preferably a pH between 5-8. Buffers suitable for use in this invention are preferably phosphate or acetate. Others such as carbonates, bicarbonates, borates, TRIS hydroxymethyl aminomethane may also be used.

Plant Extract:

The synergistic combination of the extract from the unripe fruits of the tree Tamarindus indica and a natural phenol act as a hair dye. The other parts of the plant such as leaf, flowers, ripe fruits were not found to be useful to have this effect.

Cationic Polymers:

The cationic polymer used here is preferably polyethylenimine. A wide variety of cationic polymers are known in the art and can be effectively utilized for the process of the invention. Some of these include polymers of dialkyl/trialkyl ammonium alkyl(meth)acrylate, polysaccharides or ethers or esters of polysaccharides with ammonium side groups, especially guar, cellulose and starch derivatives, polymeric adducts of ethylene oxide with ammonium groups, quaternary ethylenimine polymers, poly (dimethylaminoethyl methacrylate) or copolymers of the same with other acrylates, quaternized poly 1-vinylimidazole, etc.

Optional Ingredients:

Various optional ingredients such as thickeners, organic solvents, surface-active agents, pH adjusting agents, antioxidants, perfumes etc may be employed in the composition.

The organic solvents are included in the composition as co-solvents to assist dissolution of the components of the composition. The level of the organic solvent is preferably 1-10% by weight. Suitable solvents are mono and poly hydric alcohols such as ethyl alcohol, isopropyl alcohol, propylene glycol, benzyl alcohol etc.

Surfactants employed in the composition can be anionic, cationic, nonionic, amphoteric or zwitterionic. To mention a few examples they may be selected from higher alkylbenzene sulphonates, alkylnaphthalene sulphonates, sulphonated esters of alcohols and poly basic acids, taurates, fatty alcohol sulphates, sulphates of branched chain or secondary alcohols, salts of fatty acids or fatty acid mixtures, N-oxyalkylated fatty acid alkanolamides, and others. The surface active agents may be selected from those given in the well-known textbooks “Surface Active Agents”, Volume I by Schwartz and Perry and “Surface Active Agents and Detergents”, Volume II by Schwartz, Perry and Berch. The levels may be from about 0.05 to 15% by weight. The anionic and nonionic surfactants are used as emulsifiers and the cationic surfactants are useful as conditioning agents.

A thickening agent may also be incorporated to increase the viscosity of the formulation. This will enable easy application of the composition on the hair. Thickening agents are selected from sodium alginate, gums, or cellulose derivatives. The quantity to be incorporated will be in the range 0.1 to 20% by weight and the preferred viscosity of the formulation is in the range 100 cps to 10,000 cps.

Certain metal ions such as copper, zinc, nickel, cobalt and iron when present in the composition are known to enhance colour development.

In practice the process of the present invention is conducted at room temperature for a period of up to an hour. Elevated temperatures may be used by the use of a hair dryer. At the end of the dyeing process the hair is washed using a conventional shampoo.

The product can be formulated either in the form of a gel or as a liquid. The invention will now be demonstrated by the following non-limiting examples. In the examples and throughout this specification, all percentages are by weight unless otherwise indicated.

### EXAMPLES

### Example 1

Demonstration of the Substantivity of the Dye:

Samples of white hair (20-25 strands) were bunched together and dipped in solutions containing polyethyleneimine (PEI), a cationic polymer (solution A) for 20 min and the dye solution comprising DOPA, catechol, FeCl₃, and tamarind extract in proportions as shown in Table 1 (solution B), overnight at room temperature. After an overnight incubation the hair were removed, washed twice with shampoo and dried. The colour of the hair was then recorded.

TABLE 1

| Test | Sample | Sample | Sample | Sample | Sample | Sample |
|---|---|---|---|---|---|---|
| Conditions | 1 | 2 | 3 | 4 | 5 | 6 |
| Solution | 0.00125 | — | 0.00125 | 0.00125 | — | 0.00125 |
| A (PEI) | % | | % | % | | % |
| Solution | | | | | | |
| B: | | | | | | |
| Dopa | 100 mg | 100 mg | 100 mg | — | — | — |
| Catechol | — | — | — | 100 mg | 100 mg | 100 mg |
| FeCl₃ | 15 mg | 15 mg | 15 mg | 15 mg | 15 mg | 15 mg |
| Tamarind | 1 gm | 1 gm | — | 1 gm | 1 gm | — |
| Ext. | | | | | | |
| Buffer or | To 100 | To 100 | To 100 | To 100 | To 100 | To 100 |
| water | ml | ml | ml | ml | ml | ml |
| Results | Hair | Hair | Hair | Hairs | Hair | Hair |
| | was | was | was | were | was | was |
| | evenly | coated | coated | evenly | coated | coated |
| | coated | black | black | coated | black | black |
| | black | but the | but | black | but the | but |
| | and the | dye was | were | and the | dye was | were |
| | dye was | not | very | dye was | not | very |
| | stable | substan- | patchy | stable | substan- | patchy |
| | to | tive | and not | to | tive | and not |
| | repeated | and was | evenly | repeated | and was | evenly |
| | sham- | washed | coated | sham- | washed | coated |
| | pooing | off | | pooing | off | |
| | | easily | | | easily | |

It was seen that a pre-rinse in Solution A and the tamarind extract was essential for substantivity of colour.

### Example 2

Demonstration of Variations in Colour Obtained:

By varying the concentrations of catechol, tamarind extract and the pH of the solution, it was possible to get different 5 colours as presented in Table 2.

TABLE 2

| Conc. of | | | | | | 0.1 |
|---|---|---|---|---|---|---|
| catechol | 5 mg | 5 mg | 0.1 mg | 10 mg | 0.1 mg | mg |
| pH | 6.8 | 8.0 | 8.0 | 8.0 | 8.0 | 8.0 |
| Conc. of | 10 mg | 10 mg | 10 mg | 10 mg | 10 mg | 25 mg |
| TE | | | | | | |
| Water to | 1 ml | 1 ml | 1 ml | 1 ml | 1 ml | 1 ml |
| make up | | | | | | |
| volume to | | | | | | |
| Colour | Red | Green | Grey/ | Brown | Grey/ | Brown |
| obtained | | | brown | | brown | |

### Example 3

Demonstration of the Effect of Unripe Tamarind Extract:

Spectrophotometer readings showing the formation of dopachrome (O.D. 475 nm) only with raw fruit extract and not with ripe fruit extract are as follows.

| | O.D at 475 nm | O.D at 475 nm |
|---|---|---|
| | at 5 min | at 15 min |
| Raw fruit | 1.338 | 1.737 |
| Ripe fruit | 0.045 | 0.069 |

The data presented in Table 3 shows that only when raw fruit is used significant amount of melanin was generated but not when ripe tamarind fruit was used.

Thus by way of the present invention it has been possible to formulate a composition for changing the colour of hair and also obtain a range of desired colours. The product could also be used safely as a skin darkening agent.

1. A composition for colouring hair and/or skin comprising:
(a) a cationic polymer;
(b) a natural phenol; and
(c) unripe tamarind extract.

2. A composition according to claim 1 wherein the natural phenol comprises catechol and/or DOPA and/or homologues, analogues or derivatives of these compounds.

3. A composition according to claim 1 wherein the natural phenol comprises a DOPA species selected from the group consisting of: cysteinyl dopa, alpha alkyl dopa having 1-4, preferably 1 to 2 carbon atoms in the alkyl group, epinephrine and dopa alkyl esters having 1 to 6 carbon atoms in the alkyl group.

4. A composition according to claim 1 wherein the natural phenol comprises a DOPA species present as a soluble acid salt selected from hydrochloride and sulfate salts.

5. A composition according to claim 1 wherein the natural phenol comprises a DOPA species present as a basic salt selected from alkali and alkaline earth metal salts.

6. A composition according to claim 1 wherein the natural phenol comprises a natural plant extract comprising DOPA and/or other catechols.

7. A composition according to claim 1 wherein the composition further comprises a transition metal ion selected from the group consisting of: copper, zinc, nickel, cobalt and iron.

8. A composition according to claim 7 wherein the amount of transition metal ion in the composition is from 1 to 20 mg/100 ml.

9. A composition according to claim 1 wherein the cationic polymer is present in an amount of from 0.0005 to 0.05% by wt.

10. A composition according to claim 1 wherein the natural phenol is present in a concentration of from 0.5 to 5 mg/ml.

11. A composition according to claim 1 wherein the unripe tamarind extract is present in a concentration of from 0.005 to 0.05 mg/ml.

12. A composition according to claim 1 wherein the ratio of the concentration of natural phenol to the concentration of unripe tamarind extract is in the range of from 1:100 to 1:10.

13. A composition according to claim 1 wherein (a) is separate from (b) and (c) in a two part system.

14. A composition according to claim 1 wherein parts (b) and (c) have a pH in the range of from 4 to 10, preferably in the range of from 5 to 8.

15. A composition according to claim 1 wherein the unripe tamarind extract is derived from the unripe fruits of the tree Tamarindus indica.

16. A composition according to claim 1 wherein the cationic polymer is selected from the group consisting of: polyethylenimine; polymers of dialkyl/trialkyl ammonium alkyl(meth)acrylate; polysaccharides or ethers or esters of polysaccharides with ammonium side groups, especially guar, cellulose and starch derivatives; polymeric adducts of ethylene oxide with ammonium groups; quaternary ethyleneimine polymers; poly (dimethylaminoethyl methacrylate) or copolymers of the same with other acrylates; and quaternized poly 1-vinylimidazole.

17. A kit of parts suitable for forming a composition according to claim 1 comprising a cationic polymer packaged separately from a natural phenol and an unripe tamarind extract.

18. A method for dyeing hair comprising the steps of:
(i) mixing a natural phenol, an unripe tamarind extract and a transition metal ion in a buffered system with a pH in the range of from 4 to 10;
(ii) applying an aqueous solution of a cationic polymer on to the hair;
(iii) after step (ii) applying the mixture described in (i) on to the hair; and
(iv) allowing the said mixture to remain in contact with the hair for a sufficient time to permanently colour the hair.

19. Use of a composition according to claim 1 or a kit of parts according to claim 17 for colouring hair and/or skin.
